(19) United States
(12) Patent Application Publication
(10) Pub. No.: US 2015/0065982 A1
(43) Pub. Date: Mar. 5, 2015

(54) ARRAY OF DISPOSABLE ABSORBENT ARTICLES WITH IMPROVED PERFORMANCE THROUGH PLACEMENT OF THE ACQUISITION/DISTRIBUTION SYSTEM

(71) Applicant: The Procter & Gamble Company, Cincinnati, OH (US)

(72) Inventors: Raymond Scott HAMILTON, Lebanon, OH (US); Bret Darren SEITZ, West Chester, OH (US); Luke Robinson MAGEE, Cincinnati, OH (US)

(21) Appl. No.: 14/309,129

(22) Filed: Jun. 19, 2014

Related U.S. Application Data
(60) Provisional application No. 61/840,141, filed on Jun. 27, 2013.

(51) Int. Cl. A61F 13/551

(52) U.S. Cl. CPC A61F 13/55105; A61F 2013/530481
USPC 604/385.02

(57) ABSTRACT

An array of packages comprising three or more different sizes of absorbent articles, the array comprising a first, a second and a third packages comprising first, second and third absorbent article having first, second and third sizes. The Array Acquisition/Distribution System to Product Pitch Ratio of the first, second, and third packages is from about 10% to about 28%.

### CROSS REFERENCE TO RELATED APPLICATION

This application is a continuation of, and claims priority to U.S. Patent Application Ser. No. 61/840,141, filed on Jun. 27, 2013, the entire disclosure of which is hereby incorporated by reference.

### FIELD

The present disclosure relates to an offering of an array of disposable absorbent articles designed for improved performance through the placement of the acquisition/distribution system.

### BACKGROUND

Adult incontinence (“AI”) articles are designed to absorb and contain liquid and other discharges from the human body to prevent the body and clothing from becoming soiled. Adult Incontinence articles come in a variety of designs, each typically available in multiple sizes. The size of the articles typically affects, for example, the length or “pitch” if the article, the length and placement of the absorbent core relative to the front edge of the article, and the length and placement of the acquisition/distribution system relative to the front edge of the article. The placement of the core and acquisition/distribution system should be ideally located in close proximity to the urine exit point (also called, “pee point”) from the body so as to facilitate the article's performance. If the urine upon exit from the body does not strike the acquisition/distribution system, the probability of leakage from the product is increased.

Currently, adult incontinence articles are sold in packages that are labeled with a “size.” For example, the packages may be labeled: “Small/Medium;” “Large” or “Extra Large.” Today, AI products are marketed and sold to consumers in arrays of up to 3 sizes. This array of sizes is typically intended to cover the full range of adults. This range can span up to 300 pounds (“Is”) (from less than 100 lbs to over 400 lbs), and also over about 40 inches in waist & hip circumference (from about 25 inches to over 64 inches). By comparison, a typical array of baby diapers is available in 7 sizes, which is intended to cover a range of only about 40 lb and waist & hip circumferences of less than about 20 inches (preemie to toddler). Unfortunately, to date, offering an equally large number of sizes for AI products has not been considered due to incremental costs of these products and retailer shelf space. These barriers can be overcome by a compelling consumer need which the present invention delivers which is significantly improved fit which provides unprecedented comfort and leakage protection.

Ideally a product array should fit the broadest range of consumers with the fewest number of size offerings. Typically AI packages are labeled with a recommended wearer waist circumference range that the article is intended to fit. As a result, the waist circumference is often the sole criteria used to identify the size of an AI article. The waist size does not adequately describe the body shape of the individual and therefore does not help define the urine exit point from the body nor the product pitch and placement of the core and acquisition/distribution system that may be needed to provide the proper fit and urine handling of the article. This is the case even though other characteristics and anthropometric attributes of potential wearers (for example, age, height, weight, thigh circumference, rise and urine exit point) may vary widely within the recommended waist circumference range, and may result in an ill-fitting, ill-performing article even though a wearer's waist circumference falls within that range.

Further complicating the typical situation with respect to the identification and selection of size-appropriate articles is the presence of an overlap, from one “size” to the next, of the recommended waist circumference ranges on the associated packages. For example, a “Size S/M” article may have a recommended waist circumference range from about 28 to 40 inches, a “Size L” article may have a recommended waist circumference range from about 38 to 50 inches, and a “Size XL” article may have a recommended waist circumference range from about 48 to 64 inches. Such overlaps and lack of further definition of the body shape of the user may lead to ill-fitting articles.

Thus, there is a need for an array of articles which maximizes the product fit and placement of the urine acquisition/distribution system over all key body anthropometrics with the fewest number of sizes by modifying the product geometry.

Offering a smaller number of sizes and still achieving proper fit and performance, however, is challenging for many reasons, including that, obesity in the U.S. has doubled in the past 30 years. National Center for Health Statistics (NCHS) shows that greater than 35% of U.S. adults are considered obese. And, as weights get higher, other body anthropometrics get larger, though not proportionally. More of the population suffers from incontinence due to obesity, disease, and sports related issues. And, to make things even more challenging, as people age, their fit preferences change (e.g., younger women like lower fitting bikini style panties, whereas older women prefer higher fitting briefs).

Thus, it is one of the objects of the present disclosure to share how to meet the needs of the broad range of wearers with the fewest number of size offerings. As there is a relationship between: the consumer perceptions of fit; the range of consumer anthropometrics; the geometry and material properties of the article; and number of sizes available in an array, it is an object of the present disclosure to leverage acquisition/distribution system placement to achieve proper fit and performance across the largest range of adult consumers with the fewest size offerings.

While a lot of this specification focuses on AI products, the teaching herein may be useful for baby diapers. Particularly, it may be desirable to have a baby diaper having Array Acquisition/Distribution System to Product Pitch Ratio is from 10% to 28%, from 15% to 25%, from 18-22%, or from 19% to 21%.

### BRIEF DESCRIPTION OF THE DRAWINGS

FIG. 1 is a perspective view of an exemplary disposable pull-on garment in a typical in-use configuration;

FIG. 2 is a perspective view of an exemplary disposable pull-on garment in a typical in-use configuration;

FIG. 3 is a plan view of the pull-on garment in its flat uncontracted condition showing the inner surface;

FIG. 4A is a schematic cross section view of a third embodiment taken along line 4-4 in FIG. 3 of an exemplary disposable pull-on garment;

FIG. 4B is a schematic cross section view of a fourth embodiment taken along line 4-4 in FIG. 3 of an exemplary disposable pull-on garment;

FIG. 4C is a schematic cross section view of an eight embodiment taken along line 4-4 in FIG. 3 of an exemplary disposable pull-on garment;

FIG. 5A is a schematic cross section view taken along line 5-5 in FIG. 3 of an example of a folded outer leg cuff suitable in one embodiment of the invention;

FIG. 5B is a schematic cross section view taken along line 5-5 in FIG. 3 of an alternative embodiment example of a folded leg cuff suitable in one embodiment of the invention;

FIG. 6 is a schematic showing the coronal plane of an adult female;

FIG. 7 is a schematic showing the coronal plane of an adult female wearing an incontinence article; and

FIG. 8 is a plan view of a pull-on garment in its flat uncontracted condition showing an absorbent core and an acquisition/distribution system.

FIG. 9 is an illustration of cumulative frequency of navel to article edge for an example array of products.

FIG. 10 is an illustration of cumulative frequency of navel to vulva for an example array of products.

### DETAILED DESCRIPTION

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and to be recycled, composted or otherwise disposed of in an environmentally compatible manner). The pull-on garment may be “absorbent” such that it absorbs and contains the various exudates discharged from the body.

As used herein, the term “absorbent article” refers to pull-on garments worn by infants and other incontinent individuals, including adults, to absorb and contain urine, feces and/or menses. It should be understood, however, that the term absorbent article is also applicable to other garments such as training pants, incontinent briefs, feminine hygiene garments or panties, and the like.

As used herein, the term “acquisition/distribution system” 206 refers to a structure or structures disposed intermediate between the topsheet 58 and absorbent core 62 and is designed to facilitate the transfer of urine from the body to the absorbent core 62. These materials can consist of but are not limited to: pulp structures; non-woven materials; aperatured films; tissue; or combinations thereof. Details of the acquisition/distribution system are described in greater detail below.

As used herein, the term “rise” 203 refers to the coronal distance along the center of the female body from the navel (omphalion) to the small of the back 202, which lies in the coronal plane 207 on the same transverse horizontal plane 209 as the navel 200.

As used herein, the term “navel to vulva” refers to the coronal distance along the center of the female body from the navel (omphalion) 200 to first contact with the vulva 201.

As used herein, the term “navel to article edge” refers to the coronal distance along the center of the female body from the navel (omphalion) 200 to the first waist edge (front) of the article 134. If the first waist edge 134 is vertically below the navel 200, its value is considered negative. If the first waist edge 134 is vertically above the navel 200, its value is considered positive.

As used herein, the term “acquisition/distribution system placement from front edge” is the product distance from the first waist (front) of the article 134 to the leading edge 208 of the acquisition/distribution system 206.

As used herein, the term “product pitch” is the longitudinal length of the article from the first edge 134 to the second edge 138.

As used herein, the term “acquisition/distribution system to product pitch ratio” is the acquisition/distribution system placement from the front edge” divided by the “product pitch” and is expressed in terms of a percentage.

As used herein, the term “array acquisition/distribution system to product pitch ratio” is the average “acquisition/distribution system to product pitch ratio” from all products in an array.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “array” means a display of packages comprising disposable articles of different sizes having like article constructions (e.g., same elastomeric materials [compositionally and/or structurally] in the flaps, graphic elements) said packages having the same brand and/or sub-brand, and said packages oriented in proximity to each other in a given area of a retail store. An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Depend,” and same sub-brand, for example, “for Women Underwear.” A different array may have the brand “Depend” and the sub-brand “Silhouette For Women.” The differences between the “for Women Underwear” array and the “Silhouette For Women” arrays include different elastomeric materials in the side flaps, where “for Women Underwear” comprises strands as the elastomeric material and “Silhouette For Women” comprises a film elastomeric material.” Furthermore, the packaging is distinctly different in that “for Women Underwear” is packaged in a predominately green, film bag and “Silhouette For Women” is packaged in a predominately maroon box.

Further regarding “arrays,” as another example of two separate “arrays” having the same brand, “Certainty,” one line-up has the sub-brand “Women's Underwear.” A different array may have the same brand “Certainty” and the sub-brand “Smooth Shape Briefs for Women.” The differences between the “Women's Underwear” array and the “Smooth Shape Briefs for Women” arrays include different elastomeric materials in the side flaps, where “Women's Underwear” comprises strands as the elastomeric material and “Smooth Shape Briefs for Women” comprises a film elastomeric material.” Furthermore, the packaging is distinctly different in that “Women's Underwear” is packaged in a predominately blue, film bag and “Smooth Shape Briefs for Women” is packaged in a predominately maroon box.

Arrays also often have the same trademarks, including trademarks of the brand, sub-brand, and/or features and/or benefits across the line-up.

As used herein, the term “on-line array” means an “array” distributed by a common on-line source.

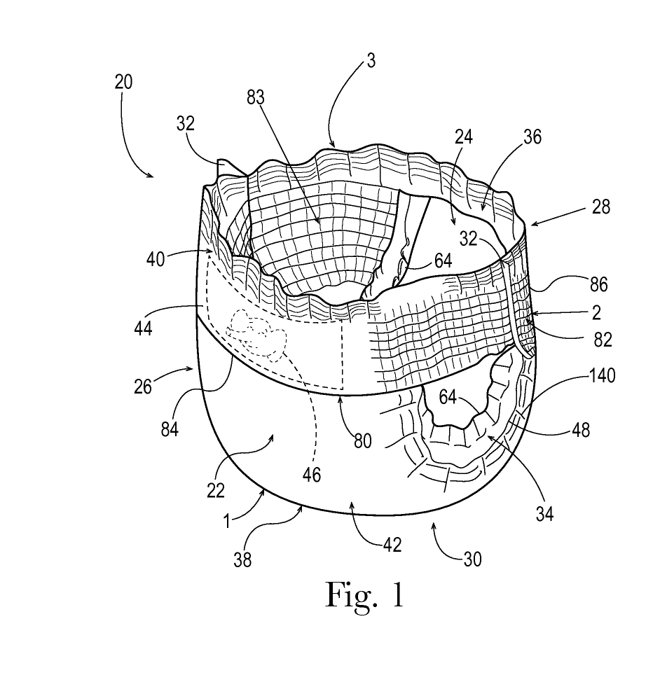

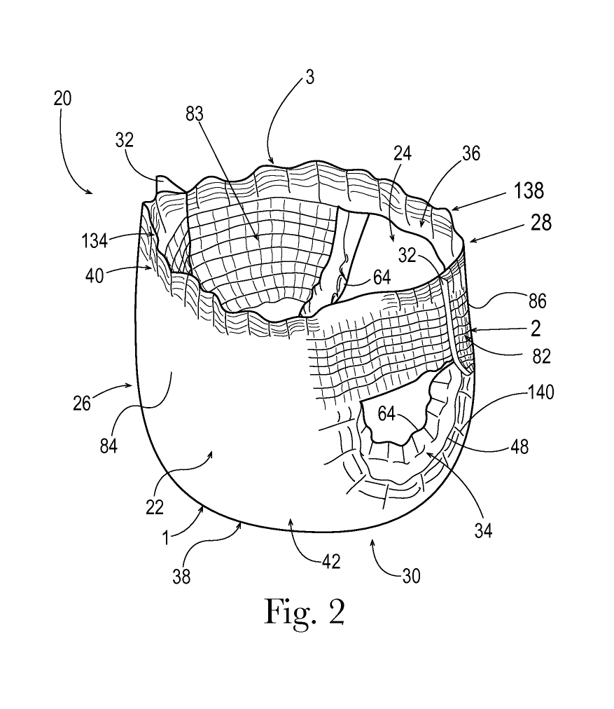

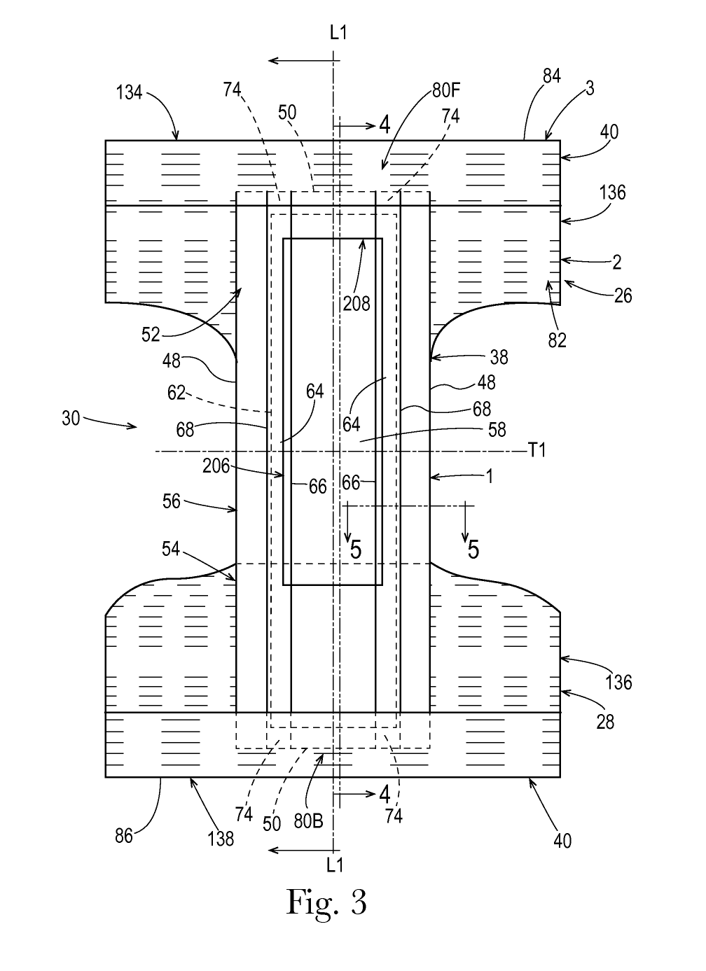

FIGS. 1 and 2 are perspective views of the absorbent article 20. As shown in FIG. 3, the absorbent article 20 has a longitudinal centerline L1 and a transverse centerline T1. The absorbent article 20 has an outer surface 22, an inner surface 24 opposed to the outer surface 22, a front region 26, a back region 28, a crotch region 30, and seams 32 which join the front region 26 and the back region 28 to form two leg openings 34 and a waist opening 36. Also referring to FIGS. 1-3, the absorbent article 20 comprises a main portion 1, a side portion 2, and a waist portion 3.

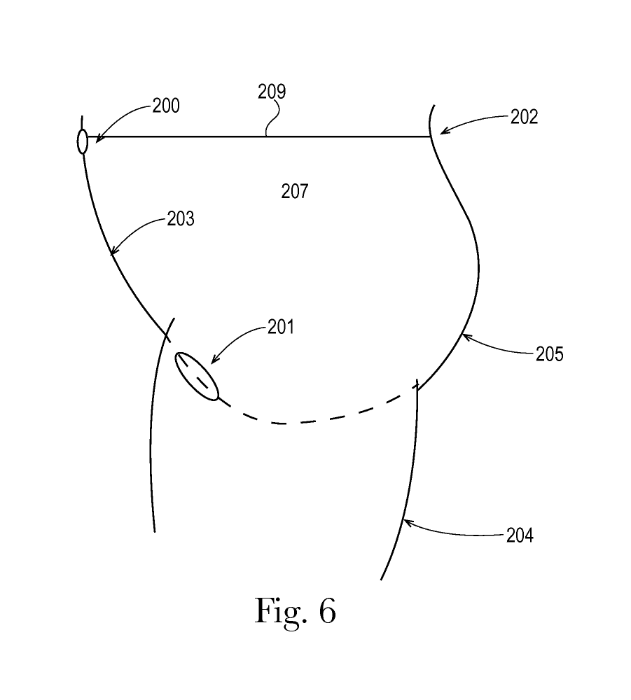

In the embodiment shown in FIGS. 1 and 3, the absorbent article 20 comprises an absorbent main body 38 (hereinafter may be referred to as “main body” or “central chassis”) to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. As shown in FIG. 3 and FIG. 6, the portions of the belts making up leg openings 34 may be shaped. While FIG. 3 illustrates discrete first belt layers 83, FIG. 6 illustrates a full outer cover nonwoven first belt layer 82.

In the embodiment shown in FIG. 2 the absorbent article 20 comprises an absorbent main body 38 to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. One or more of the belt layers may extend from a first waist edge 134 in a first waist region 26 through the crotch region to a longitudinally opposing second waist edge 138 in a second waist region 28 and may form a portion or the whole of the outer surface of the absorbent article 20.

The absorbent main body 38 absorbs and contains body exudates disposed on the main body 38. In the embodiment shown in FIG. 3, the main body 38 has a generally rectangular shape having a longitudinal centerline L, a transverse centerline T1, left and right longitudinally extending side edges 48 (hereinafter may be referred to as “longitudinal side edge”) and front and back transversely extending end edges 50 (hereinafter may be referred to as “transverse end edge”). The main body 38 also has waist panels (i.e., a front waist panel 52 positioned in the front waist region 26 of the absorbent article 20 and a back waist panel 54 positioned in the back waist region 28) and a crotch panel 56 in the crotch region 30 between the front and back waist panels 52, 54.

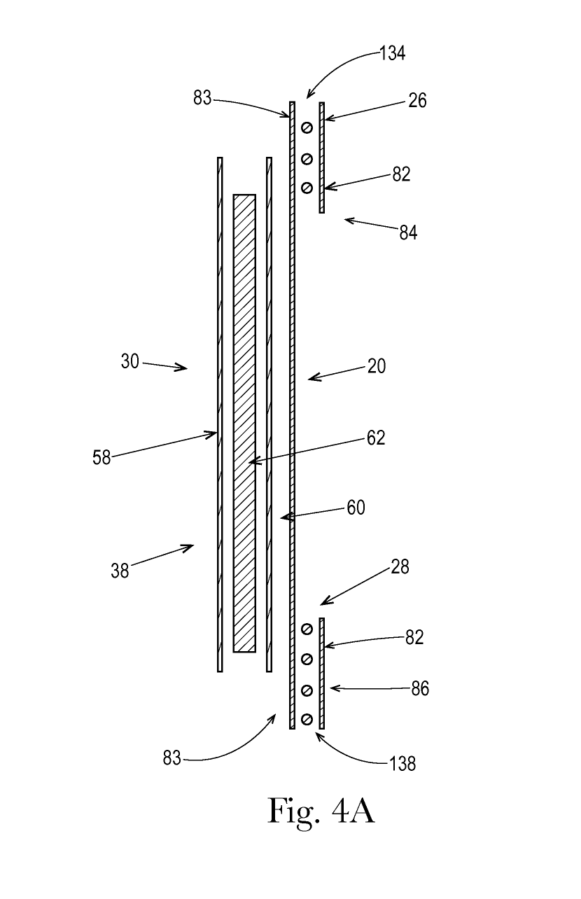

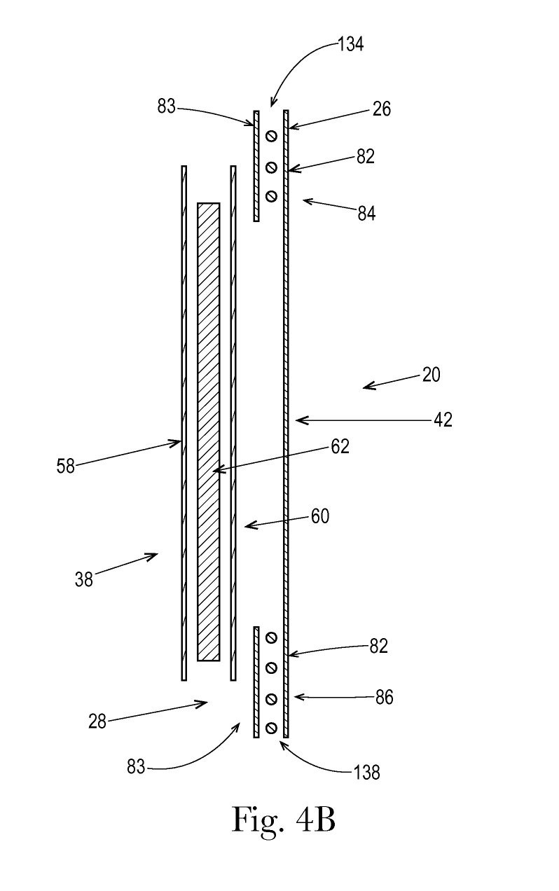

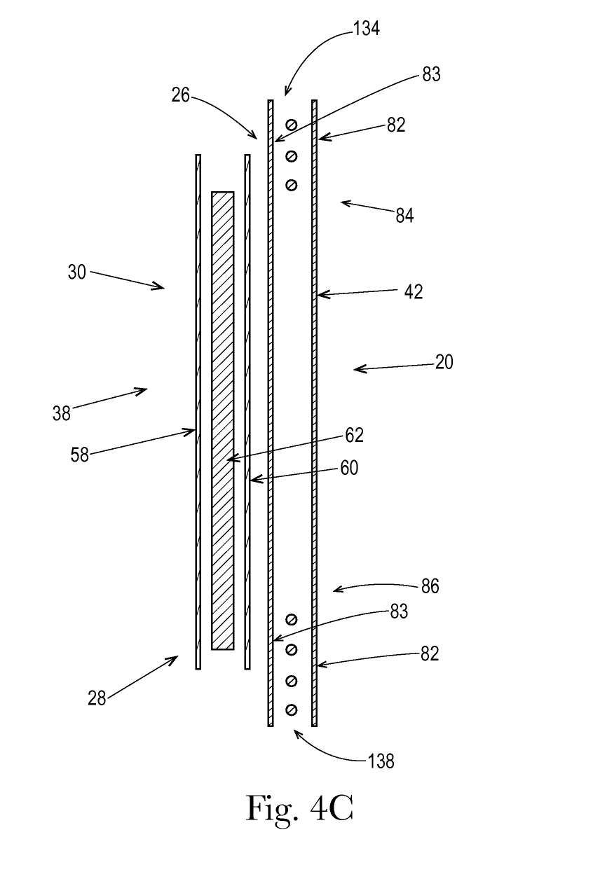

In the embodiment shown in FIGS. 4A and 4B, the absorbent articles 20 may comprise front and rear extensible belts 84, 86 disposed in the front and rear waist regions 26, 28 respectively and intended to encircle at least a portion of the waist of the wearer, the front and rear belts 84, 86 being connected by the main body that forms the crotch region 30 of the article. The first and second belt may be formed from a first belt layer 82 (e.g., a “full outer cover nonwoven” or an “outer cover web”) extending from a first waist edge 134 in a first waist region 26 through the crotch region to a longitudinally opposing second waist edge 138 in a second waist region 28 and forming a portion of the outer surface of the absorbent article 20. The front and rear belts 84, 86 also may comprise a second belt layer 83a and b (e.g., an “inner belt web”) forming a portion of the inner surface 24 of the absorbent article, the second belt layer may be formed of two longitudinally spaced webs of material. The first and second belt portions may also comprise an elastomeric material 200 (e.g., “elastic elements” or “elastics”) disposed between the first and second belt layers. The elastomeric material may comprise elastic strands, elastomeric films, elastomeric ribbons, elastomeric nonwovens, elastomeric filaments, elastomeric adhesives, elastomeric foams, scrims or combinations thereof. The main body 38 (“central or center chassis”) of the absorbent article may comprise an outer surface 22, backsheet 60, an inner surface 24, topsheet 58, and an acquisition/distribution system 206 disposed between the topsheet 58 and absorbent core 62. The first belt layer may form a portion of the outer surface 22. In addition, the main body may comprise elasticized barrier leg cuffs 64 disposed at or adjacent the side edges of the main body. The second belt layer may overlap at least a portion of the main body and one or both of the second belt layer webs may form the outer surface of the first belt layer or alternatively the inner surface of the first belt layer. Alternatively, the front portion and/or the rear portion of the first belt layer 82 may be folded along the waist edge of the belt region to wrap the elastomeric material and form a portion of the second belt layer of one or both of the front and rear belt portions 84, 86. In other words, the inner surface and outer surface of each of the belt portions may be formed from a single web of material. FIG. 6 is plan view of the embodiment consistent with FIG. 4B.

In the embodiment shown in FIG. 4C, the absorbent articles 20 may comprise front and rear belts 84, 86 intended to encircle at least a portion of the waist of the wearer, the front and rear belts 84, 86 being connected to a main body 38 forming a portion of the crotch region 30 of the absorbent article 20. The front and rear belts 84, 86 are formed from a first belt layer 82 forming a portion of the outer surface of the absorbent article. The front and rear belt portions 84, 86 also comprise a second belt layer 83 forming a portion of the inner surface 24 of the absorbent article 20. The second belt layer may be laterally discontinuous and spaced apart in a transverse direction. The first and second belt layers 82, 83 may be formed of substantially the same material or may comprise different materials. The first and second belt layers 82, 83 may be formed from nonwovens, films, foams or combinations thereof. The front and rear belt portions 84, 86 may also comprise an elastomeric material disposed between the first and second belt layers 82, 83. The elastomeric material may comprise elastic strands, elastomeric films, elastomeric ribbons, elastomeric nonwovens, elastomeric filaments, elastomeric adhesives, elastomeric foams, scrims or combinations thereof. A portion of the elastomeric material may be directly combined with the outer cover layer. The main body 38 of the absorbent article may comprise an outer surface 22, backsheet 60, an inner surface 24, topsheet 58, an acquisition/distribution system 206 disposed between the topsheet 58 and absorbent core 62, and an absorbent core 62 disposed between the topsheet 58 and the backsheet 60. In certain embodiments the backsheet may be a nonwoven and film laminate wherein the nonwoven is formed by the outer cover layer. In addition, the main body 38 may comprise elasticized barrier leg cuffs 64 disposed at or adjacent the side edges of the main body 38. The front and rear belts 84, 86 overlap at least a portion of the main body 38 and one or both of the belts may be disposed on the outer surface of the main body 38. A portion of the second belt layer and/or a portion of the first belt layer may be directly attached to the outer cover layer. The front and rear belts 84, 86 may be formed from a first belt layer extending from a first waist edge 134 in a first waist region 26 through the crotch region to a second waist edge 138 in a second waist region 28 and forming a portion of the outer surface of the absorbent article 20. The front and rear belts 84, 86 may also comprise a second belt layer extending from a first waist edge 134 in a first waist region 26 through the crotch region to a second waist edge 138 in a second waist region 28 and forming a portion of the inner surface of the absorbent article 20. The first and second belt layers may be formed of substantially the same material or may comprise different materials. The first and second belt layers may be formed from nonwovens, films, foams, woven materials or combinations thereof. The front and rear belt portions 84, 86 may also comprise an elastomeric material disposed between the first and second belt layers in one or both of the first and second waist regions 26, 28. The elastomeric material may comprise elastic strands, elastomeric films, elastomeric ribbons, elastomeric nonwovens, elastomeric filaments, elastomeric adhesives, elastomeric foams, scrims or combinations thereof. The main body 38 of the absorbent article 20 may comprise an outer surface 22, backsheet 60, an inner surface 24, topsheet 58, an acquisition/distribution system 206 disposed between the topsheet 58 and absorbent core 62, and an absorbent core 62 disposed between the topsheet 58 and the backsheet 60. One or both of the first and second belt layers may form a portion of the outer surface 22. In addition, the main body 38 may comprise elasticized barrier leg cuffs 64 disposed at or adjacent the side edges of the main body 38. A portion of one or both of the front and rear belts 84, 86 may overlap at least a portion of the main body 38. Alternatively, the front belt portion and rear belts 84, 86 may comprise a belt layer forming a first surface of the belt portion wherein the belt layer may be folded along the waist edge of the belt portion to wrap the elastomeric material and overlap a portion of the opposing belt layer. In other words, a portion of the inner surface and a portion of the outer surface of each of the belt portions may be formed from a single web of material.

Additional lateral extensibility in the main body 38 may be provided in a variety of ways. For example, a material or materials from which the main body 38 is made may be pleated by any of many known methods. Alternatively, all or a portion of the main body 38 may be made of a formed web material or a formed laminate of web materials like those described in U.S. Pat. No. 5,518,801 issued on 21 May 1996 in the name of Chappell et al. This formed web material includes distinct laterally extending regions in which the original material has been altered by embossing or another method of deformation to create a pattern of generally longitudinally oriented alternating ridges and valleys and also includes laterally extending unaltered regions between the laterally extending altered regions. The formed web material can be extended in a direction perpendicular to the ridges up to the point where the ridges and valleys flatten with substantially less force than is required to extend beyond that point. In addition to lateral extensibility, the creation of a formed laminate web as described above provides a main body 38 backsheet with improved texture and cloth-like appearance and feel. The deformation creates a cloth-like pattern in the film and increases the loft of the nonwoven in multi-layer film and nonwoven laminate backsheets.

Alternatively, a portion of the absorbent article can be ring-rolled and thus rendered highly extensible as described in U.S. Pat. No. 5,366,782 (issued Nov. 22, 1994 to Curro, et al). Specifically, a ring-rolling apparatus includes opposing rolls having intermeshing teeth that incrementally stretch and thereby plastically deform the material forming the absorbent article (or a portion thereof) thereby rendering the article extensible in the ring-rolled regions. In one embodiment, the absorbent article can be ring-rolled in a portion of at least one of the front or back waist regions, for example the portion of the main body 38 underlying and/or immediately adjacent one or both of the front and back belts 84, 86, while other regions may comprise a structured elastic-like formed web material. The article may be ring-rolled across the entire width in one or both of the waist regions or alternatively may be ring-rolled over only a portion of the main body 38 width or over only a portion of one or both of the belts.

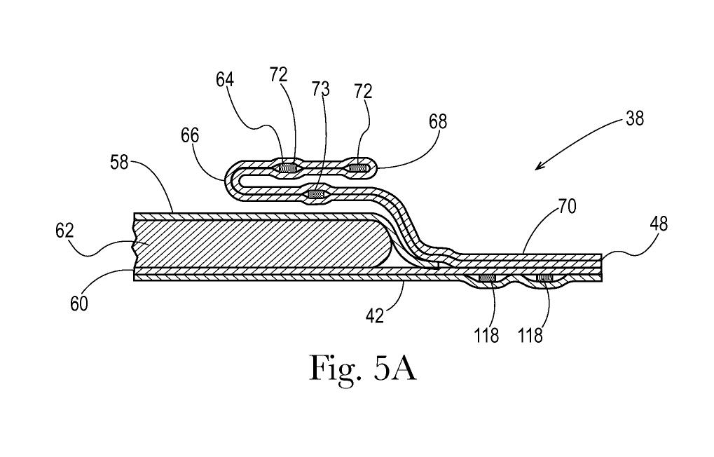

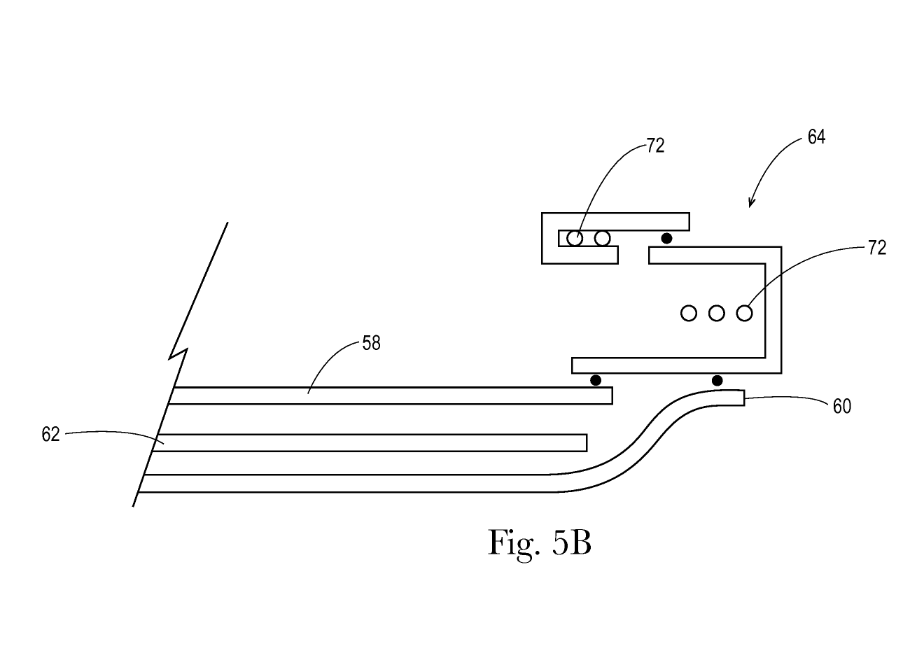

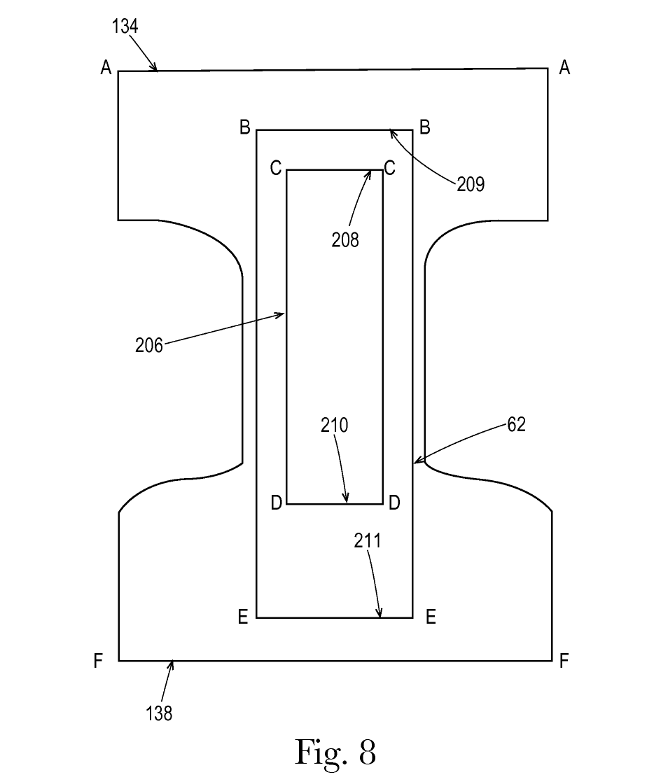

The main body 38 may comprise a liquid pervious topsheet 58, a liquid impervious backsheet 60 and an acquisition/distribution system 206 and absorbent core 62 disposed therebetween. The main body 38 may additionally comprise a barrier leg cuff 64 disposed along the longitudinal side edge 48. The barrier leg cuff 64 provides improved containment of liquids and other body exudates in the crotch region 30. The barrier leg cuff 64 shown in FIG. 5 comprises a single layer of material which may be folded to form a barrier leg cuff having two layers. The barrier leg cuff 64 extends from the side of the main body at or adjacent the longitudinal side edge 48 toward the longitudinal centerline L2. The barrier leg cuff may be folded along the folding line 66 back toward the longitudinal side edge 48. The barrier leg cuff 64 may have a first barrier cuff elastic material 72 adjacent to the distal portion 68 and a second barrier cuff elastic material 73 adjacent to the proximal portion 70 of the barrier leg cuff 64. The proximal portion 70 of the barrier leg cuff 64 may be joined to the backsheet 60 adjacent to the longitudinal side edge 48. The portion of the barrier leg cuff 64 along the folding line 66 and the distal portion 68 may be free from attachment to any portion of the main body 38 in the crotch region 30 such that the barrier leg cuff 64 stands up toward the wearer's body. The transverse end 74 of the barrier leg cuff 64 may be joined to the topsheet 58 at or adjacent the longitudinally opposing ends of the leg cuff by an attachment means which may be any known means such as an adhesive, heat bond, pressure bond or the like as shown in FIG. 5A. Examples of acceptable cuffs 64 are disclosed in U.S. Ser. No. 13/457,521, filed Apr. 27, 2012, including the configurations disclosed by FIGS. 8a-t. For, instance, as illustrated in FIG. 5B, the barrier leg cuff may be a two-piece cuff. And, the cuff 64 may be joined to the backsheet with a no leak bead 215 that runs along the entire longitudinal length of the cuff and/or the backsheet film 60.

The liquid pervious topsheet 58 may be positioned adjacent the body-facing surface of the absorbent core 62 and may be joined thereto and/or to the backsheet 60 by any attachment means known in the art. The liquid impervious backsheet 60 is generally that portion of the absorbent article 20 positioned adjacent the garment-facing surface of the absorbent core 62 and prevents the exudates absorbed and contained therein from soiling articles that may contact the absorbent article 20. The absorbent core is positioned between the topsheet 58 and the backsheet 60 and absorbs and retains liquids such as urine and other certain body exudates.

The topsheet 58, the backsheet 60 and the absorbent core may be manufactured any known materials. Suitable topsheet materials may include porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Suitable backsheet materials may include breathable materials that permit vapors to escape from the diaper while still preventing exudates from passing through the backsheet.

A suitable absorbent core for use in the absorbent article 20 may comprise any absorbent material which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). In some embodiments, the absorbent core may comprise a fluid acquisition component, a fluid distribution component, and a fluid storage component. An example of a suitable absorbent core having a fluid acquisition component, a fluid distribution component, and a fluid storage component is described in U.S. Pat. No. 6,590,136. Examples of acceptable air felt free cores (i.e., absorbent core systems having little or no air felt) are disclosed in U.S. Pat. Nos. 5,562,646, 7,750,203, 7,744,576 and U.S. Pub. Nos. 2008/0312617A1, 2008/0312619A1, and 2004/0097895A1.

A suitable acquisition/distribution system 206 may alternatively or additionally comprise an acquisition layer. The acquisition layer may be disposed, for example, immediately below the topsheet 58. The acquisition layer may be or may comprise a non-woven material, such as an SMS or SMMS material, comprising a spunbonded, a melt-blown and a further spunbonded layer or alternatively a carded chemical-bonded nonwoven. In some embodiments, the acquisition layer may comprise air or wet-laid cellulosic, cross-linked cellulosic, or synthetic fibers, or blends thereof. In certain embodiments, the acquisition layer may comprise a roll-stock web of synthetic fibers, or a combination of synthetic and cellulosic fibers, bonded together to form a highloft material. Alternatively, the acquisition layer may comprise absorbent open cell foam. The nonwoven material may be latex bonded. Example acquisition layers are disclosed in U.S. Pat. No. 7,786,341. Carded, resin-bonded nonwovens may be used, in particular where the fibers used are solid round or round hollow PET staple fibers (50/50 or 40/60 mix of 6 denier and 9 denier fibers). An example binder is a butadiene/styrene latex. Nonwovens have the advantage that they can be manufactured outside the converting line and stored and used as a roll of material. The acquisition layer 52 may be stabilized by a latex binder, for example a styrene-butadiene latex binder (SB latex).

A further acquisition layer may be used in addition to a first acquisition layer described above. For example, a tissue layer may be placed between the first acquisition layer and the distribution layer. The tissue may have enhanced capillarity distribution properties compared to the acquisition layer described above. The tissue and the first acquisition layer may be of the same size or may be of a different size. For example, the tissue layer may extend further in the rear of the absorbent article than the first acquisition layer. An example of hydrophilic tissue is a 13-15 gsm high wet strength made of cellulose fibers from supplier Havix.

The composition used to form fibers for the base substrate of the acquisition layer can include thermoplastic polymeric and non-thermoplastic polymeric materials. The thermoplastic polymeric material must have rheological characteristics suitable for melt spinning. The molecular weight of the polymer should be sufficient to enable entanglement between polymer molecules and yet low enough to be melt spinnable. For melt spinning, thermoplastic polymers have molecular weights below about 1,000,000 g/mol, preferably from about 5,000 g/mol to about 750,000 g/mol, more preferably from about 10,000 g/mol to about 500,000 g/mol and even more preferably from about 50,000 g/mol to about 400,000 g/mol. Unless specified elsewhere, the molecular weight indicated is the number average molecular weight.

The thermoplastic polymeric materials are able to solidify relatively rapidly, desirably under extensional flow, and form a thermally stable fiber structure, as typically encountered in known processes such as a spin draw process for staple fibers or a spunbond continuous fiber process. Preferred polymeric materials include, but are not limited to, polypropylene and polypropylene copolymers, polyethylene and polyethylene copolymers, polyester and polyester copolymers, polyamide, polyimide, polylactic acid, polyhydroxyalkanoate, polyvinyl alcohol, ethylene vinyl alcohol, polyacrylates, and copolymers thereof and mixtures thereof. Other suitable polymeric materials include thermoplastic starch compositions as described in detail in U.S. Pat. Pub. Ser. No. 2003/0109605A1 and 2003/0091803. Other suitable polymeric materials include ethylene acrylic acid, polyolefin carboxylic acid copolymers, and combinations thereof. Suitable polymers are described in U.S. Pat. No. 6,746,766, U.S. Pat. No. 6,818,295, and U.S. Pat. No. 6,946,506 and U.S. Pat. Pub. Ser. No. 03/0092343. Common thermoplastic polymer fiber grade materials may be used, such as polyester based resins, polypropylene based resins, polylactic acid based resin, polyhydroxyalkanoate based resin, and polyethylene based resin and combination thereof. Some embodiments use polyester and polypropylene based resins.

Nonlimiting examples of thermoplastic polymers suitable for use the acquisition/distribution system 206, including the acquisition and/or distribution layers include aliphatic polyesteramides; aliphatic polyesters; aromatic polyesters including polyethylene terephthalates (PET) and copolymer (coPET), polybutylene terephthalates and copolymers; polytrimethylene terephthalates and copolymers; polypropylene terephthalates and copolymers; polypropylene and propylene copolymers; polyethylene and polyethylene copolymers; aliphatic/aromatic copolyesters; polycaprolactones; poly(hydroxyalkanoates) including poly(hydroxybutyrate-co-hydroxyvalerate), poly(hydroxybutyrate-co-hexanoate), or other higher poly(hydroxybutyrate-co-alkanoates) as referenced in U.S. Pat. No. 5,498,692 to Noda; polyesters and polyurethanes derived from aliphatic polyols (i.e., dialkanoyl polymers); polyamides; polyethylene/vinyl alcohol copolymers; lactic acid polymers including lactic acid homopolymers and lactic acid copolymers; lactide polymers including lactide homopolymers and lactide copolymers; glycolide polymers including glycolide homopolymers and glycolide copolymers; and mixtures thereof. Additional examples include aliphatic polyesteramides, aliphatic polyesters, aliphatic/aromatic copolyesters, lactic acid polymers, and lactide polymers.

Suitable lactic acid and lactide polymers include those homopolymers and copolymers of lactic acid and/or lactide which have a weight average molecular weight generally ranging from about 10,000 g/mol to about 600,000 g/mol, from about 30,000 g/mol to about 400,000 g/mol, or from about 50,000 g/mol to about 200,000 g/mol. An example of commercially available polylactic acid polymers includes a variety of polylactic acids that are available from the Chronopol Incorporation located in Golden, Colo., and the polylactides sold under the tradename EcoPLAÒ. Examples of suitable commercially available polylactic acid are NATUREWORKS from Cargill Dow and LACEA from Mitsui Chemical. It may be desirable to have a homopolymer or copolymer of poly lactic acid having a melting temperature from about 160° to about 175° C. Modified poly lactic acid and different stereo configurations may also be used, such as poly L-lactic acid and poly D,L-lactic acid with D-isomer levels up to 75%. Optional racemic combinations of D and L isomers to produce high melting temperature PLA polymers are also desirable in some embodiments. These high melting temperature PL polymers are special PLA copolymers (with the understanding that the D-isomer and L-isomer are treated as different stereo monomers) with melting temperatures above 180° C. These high melting temperatures are achieved by special control of the crystallite dimensions to increase the average melting temperature.

Depending upon the specific polymer used, the process, and the final use of the fiber, more than one polymer may be desired. The polymers of the present disclosure are present in an amount to improve the mechanical properties of the fiber, the opacity of the fiber, optimize the fluid interaction with the fiber, improve the processability of the melt, and improve attenuation of the fiber. The selection and amount of the polymer will also determine if the fiber is thermally bondable and affect the softness and texture of the final product. The fibers of the present disclosure may comprise a single polymer, a blend of polymers, or be multicomponent fibers comprising more than one polymer. The fibers in the present invention are thermally bondable.

Multiconstituent blends may be desired. For example, blends of polyethylene and polypropylene (referred to hereafter as polymer alloys) can be mixed and spun using this technique. Another example would be blends of polyesters with different viscosities or monomer content. Multicomponent fibers can also be produced that contain differentiable chemical species in each component. Non-limiting examples would include a mixture of 25 melt flow rate (MFR) polypropylene with 50 MFR polypropylene and 25 MFR homopolymer polypropylene with 25 MFR copolymer of polypropylene with ethylene as a comonomer.

The polymeric materials may have melting temperatures above 110° C., above 130° C., above 145° C., above 160° C. or above 200° C. Polymers with high glass transition temperatures may be desired. Glass transition temperatures in the end-use fiber form may be used that are above −10° C., which are above 0° C., which are above 20° C., or that are above 50° C. This combination of properties produces fibers that are stable at elevated temperatures. Exemplary examples of materials of this type are polypropylene, polylactic acid based polymers, and polyester terephthalate (PET) based polymer systems.

As mentioned in the Background, the acquisition/distribution systems 206 is more desirably kept as small as possible and is precisely placed (versus nonspecific placement of a larger acquisition/distribution systems 206). The length of the acquisition/distribution systems 206 may range from 200 mm to 300 mm, from 210 mm to 290 mm, form 220 mm to 280 mm, from 230 mm to 260 mm, or from 240 mm to 250 mm. The width may range from 60 mm to 90 mm, from 70 mm to 80 mm, or from 73 mm to 77 mm.

Other suitable acquisition/distribution systems 206, as well as absorbent cores 62, including composition, dimension, and disposition, are described and illustrated in U.S. Pat. and Pub. Nos. 7,138,561; 7,682,686; 7,410,683; 2010-0310845; 2010-0312208; 2010-0312212; 2010-0310837; 2010-0310810; 2010-0312211; 2012-0238978; 2012-0238981; 2012-0238979; 2012-0238982; 2012-0238170; and 2012-0237718.

The outer cover layer 42 may be disposed on the outer surface 22 of the absorbent article 20 and covers the crotch panel 56 of the absorbent main body 38. The outer cover layer 42 may extend into and cover the front waist panel 52 and the back waist panel 54 of the main body 38. The outer cover layer may form a portion of the backsheet and/or the main body. The outer cover layer 42 may be directly joined to and cover a portion or all of the liquid impervious backsheet 60 of the main body 38. The central panel 80 of the front and back belt 84, 86 may be joined to the front waist panel 52 and the back waist panel 54 of the main body 38 through the outer cover layer 42. Thus, the outer cover layer 42 is disposed between the front and back belt 84, 86 and the liquid impervious backsheet 60 of the main body 38. In one embodiment shown in FIGS. 2 and 4A, the outer cover layer 42 is coextensive with the liquid impervious backsheet 60. The leg elastic material 140 is disposed so as to extend generally longitudinally along the longitudinal side edge 48 of the main body 38. The leg elastic material 140 may be disposed at least in the crotch region 30 of the absorbent article 20 or may be disposed along the entirety of the longitudinal side edge 48.

The outer cover layer 42 may comprise a material separate from the material of the inner layer 83 and the outer layer 82 constituting the belt 40. The outer cover layer 42 may comprise two or more layers of materials. The outer cover layer 42 may comprise any known materials and may comprise materials used for the front and back belt 84, 86 as explained above. The outer cover layer 42 may comprise a single layer of nonwoven web of synthetic fibers. The outer cover layer 42 may comprise a single layer of hydrophobic, non-stretchable nonwoven material. The outer cover layer may comprise a film, a foam, a nonwoven, a woven material or the like and/or combinations thereof such as a laminate of a film and a nonwoven.

The belt 40 may comprise a front belt 84 and a back belt 86 (hereinafter may be referred to as “front and back belt 84, 86) and has a ring-like configuration by permanently or refastenably connecting the front belt 84 and the back belt 86 at the seams 32 or by permanently or refastenably connecting the front and/or back belt to the main body 38. Articles of the present disclosure may have refastenable elements, configurations, and methods of making as disclosed in U.S.S.N.s 61/787,416, filed on Mar. 15, 2013, as well as U.S. Ser. No. 61/787,332, filed on Mar. 15, 2013. The refastenable elements may be fastened during the manufacturing process and/or fastened in the package prior to use by the wearer or caregiver (i.e., the pant may be sold in “closed form”). The front and back belts 84 and 86 may be in a number of configurations as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of the '065 application).

The belt 40 may be ring-like and elastic. The ring-like elastic belt 40 extends transversely about the waist opening 36 of the absorbent article 20 and acts to dynamically create fitment forces and to distribute the forces dynamically generated during wear. Applicants have found that improved fit can be created by controlling the distance between, linear density, and the pre-strain of the elastomeric material in relation to each other and to the openings for the body. This may occur by choosing different materials throughout the belt 40 that exhibit desired properties. The different materials are combined at specific distances, linear densities, and prestrains to create a belt 40 that acts to dynamically create fitment forces. Particularly, the articles of this disclosure may have the characteristics of the articles of Examples 1-4 as disclosed in U.S. Ser. No. 13/764,990, filed Feb. 12, 2013. Articles of the present disclosure may also have the same stress, strain and spacing of its elastics as disclosed in U.S. Ser. No. 13/764,990 and/or as disclosed in U.S. Ser. No. 61/598,012, filed Feb. 13, 2012. Articles of the present disclosure may also have the same elastic sections and force zones disclosed in U.S. Ser. No. 13/764,990.

The front belt 84 may comprise 5 to 50 elastic strands. The front belt 84 may comprise 10 to 20 elastic strands. The back belt 86 may comprise 5 to 50 elastic strands. The back belt 86 may comprise 10 to 20 elastic strands. The elastic strands are distributed amongst the different force zones. Elastic strands may be distributed evenly amongst the force zones. Elastic strands may also be distributed unevenly amongst the different force zones. Each force zone comprises at least one elastic strand.

The elastic strands may have a linear density between 200 to 2500. Linear density is the density of the elastic fibers in the elastic strand. The most commonly used unit for the linear density is the decitex, abbreviated “dtex,” which is the mass in grams per 10,000 meters. The linear density may be used to change the force profile. For example, one could reach a desired force profile by selecting the linear density of a single elastic strand, combining multiple elastic strands with a smaller linear density in close proximity to each other, and/or combining with other elastomeric materials.

The elastic strands may have an elastic pre-strain. The elastic pre-strain is the percent of length increase in an elastic strand or plurality of elastic strands at the point of combining the elastic(s) with the first and/or second belt layers. For example a strand with a free length of 15 centimeters (cm) may have a load applied such that the 15 cm elastic strand is now 18 cm long. This length increase of 3 cm is 20% of 15 cm ( 3/15), or a 20% strain. The elastic pre-strain may be used to change the force profile of a single elastic strand or a plurality of elastic strands. Force profiles may also be changed by changing the linear density in conjunction with the elastic pre-strain of one or more elastic strands.

The number of elastic strands in each zone may be changed according to the placement of the absorbent core. Applicants have found that the use of thinner absorbent cores may lead to a need in increased elastic force to compensate for the change in article thickness. The force profile must be adjusted depending on the location and thickness of the absorbent core. This particularly affects the second elastic section and third elastic section.

The elastic strands disposed in the belt may be aligned in a curved fashion so that a tangent of the curve of the elastic strands may form an acute angle with the centerline or may form an arcuate shape. This may allow for targeting the force profile and/or coordinating print and elastication/rugosities/elastics in the stretch sections.

The elasticized belts and rear region of the pant may be in a number of configurations as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of the '065 application).

It may be desirable to use the hot air seaming processes, as well as the article forming processes disclosed in U.S. Pat. No. 6,248,195 and U.S. Ser. Nos. 12/795,021, 13/401,907, and 13/402,056 for seaming articles as disclosed herein. And, the articles disclosed herein may have graphics in accordance with U.S.S.N.s 61/646,953 and 61/646,979, each filed on May 15, 2012.

FIG. 6 illustrates the coronal plane 207 of an adult female, the navel (omphalion) 200, the small of back 202 (on the same transverse horizontal plane 209 as the navel 202), and the “surface of the body along the coronal plane from the navel to small of back” 203. The female's vulva is denoted by 201, the buttocks by 205 and leg by 204.

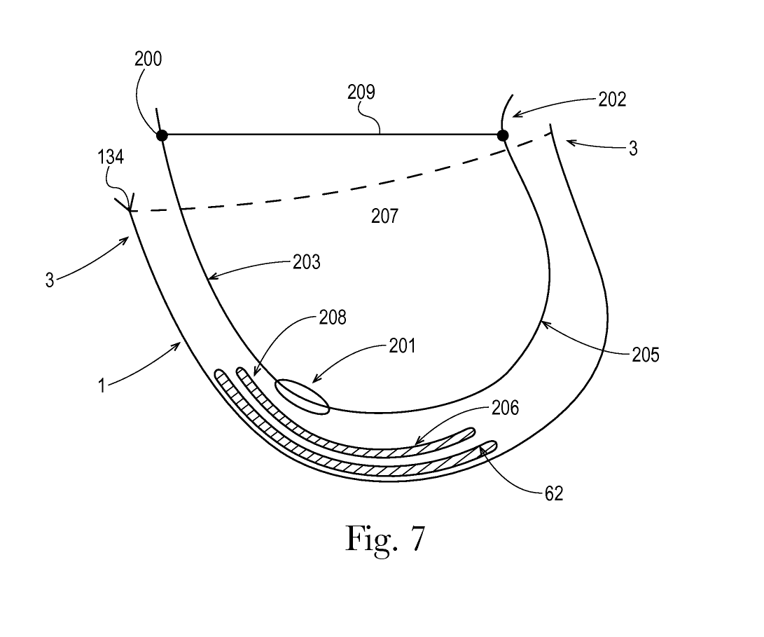

FIG. 7 illustrates the coronal plane 207 of an adult female wearing an incontinence article and also illustrates the navel (omphalion) 200, the small of back 202 (on the same transverse horizontal plane 209 as the navel), the “surface of the body along the coronal plane from the navel to small of back” 203, the female's vulva 201, and the buttocks 205. The article comprises a first (front) waist edge 134, an absorbent core 62 and an acquisition/distribution system 206.

FIG. 8 illustrates the plan view of a disposable article, showing the absorbent core 62 and acquisition/distribution system 206. The first waist edge (front) 134 of the article is defined by line A-A. The absorbent core's first edge (front) 209 is defined by line B-B. The acquisition/distribution system's first edge (front) 208 is defined by line C-C. The acquisition/distribution system's second edge (back) 210 is defined by line D-D. The absorbent core's second edge (back) 211 is defined by line E-E. The second waist edge (back) 138 is defined by line F-F.

It may be desirable to position the acquisition/distribution system 206 (acquisition/distribution system placement from front edge) such that it covers the vulva 201 in order to facilitate urine acquisition and distribution/transfer into the absorbent core 62. The range and distribution of where the vulva lies relative to the navel (navel to vulva), and also the range and distribution of where the product sits on the body relative to the navel (navel to article edge) are factors in accomplishing proper design to achieve proper fit and performance.

The range for “navel to article edge” delivered by currently marketed adult incontinence articles as worn by the population of adults is typically from about −6 cm (below the navel) to about 6 cm (above the navel). When navel to article edge values fall below 6 cm, the consumer may believe the product is too small/short, and if above 6 cm, the consumer may believe the product is too large/long. Consumers may purchase and wear products that fall within this range, and switch to a smaller or larger size accordingly.

If each product within an array is designed and targeted to a specific waist circumference range, then the range for the navel to vulva distance for each product can be also predicted as anthropometric data exists which links individual consumers various anthropometric measures. One example of an inventive product array, showing the product pitch, the targeted waist circumference range and the predicted range for navel to vulva distance (from existing anthropometric data) is shown on table 1.

TABLE 1
Example Product Array

| Product Array | Example Product |
|---|---|
| Number of Packages in Array | 3 |
| First Package in Array | S/M |
| Product Pitch (cm) | 72 |
| Target Waist Circumference | 71-102 |
| Range (cm) | |
| Predicted Range of Navel to | 12-30 |
| Vulva (cm) | |
| Second Package in Array | L |
| Product Pitch (cm) | 79 |
| Target Waist Circumference | 97-127 |
| Range (cm) | |
| Predicted Range of Navel to | 15-35 |
| Vulva (cm) | |
| Third Package in Array | XL |
| Product Pitch (cm) | 82 |
| Target Waist Circumference | 122-163 |
| Range (cm) | |
| Predicted Range of Navel to | 19-37 |
| Vulva (cm) | |

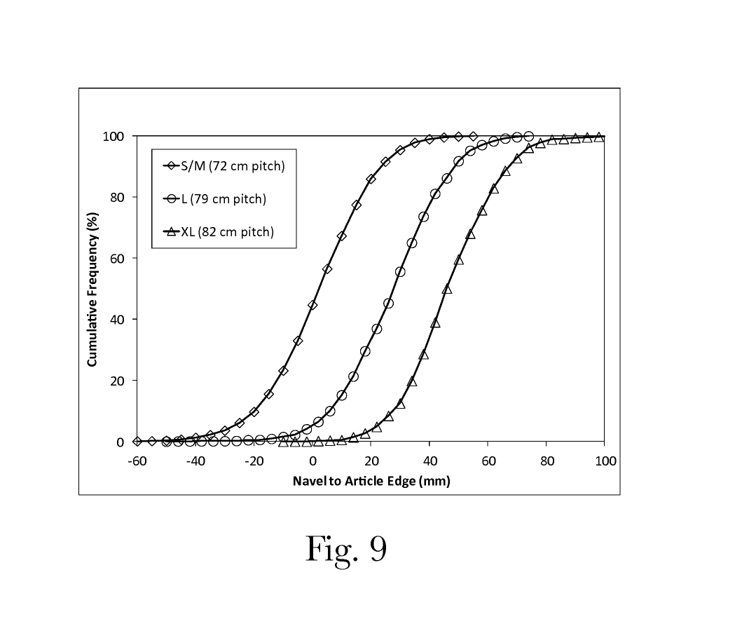

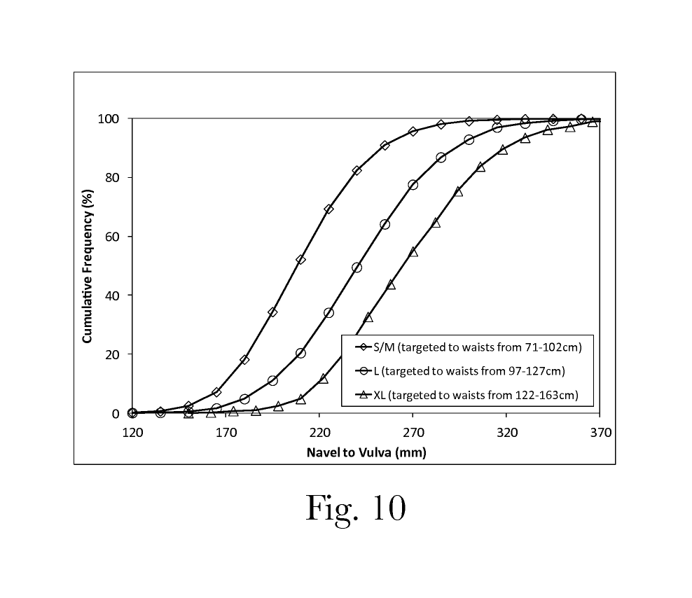

For such a product array, it may be determined through consumer testing and product modeling where the product sits on the adult body (navel to article edge). The cumulative frequency for each size may be represented as shown in FIG. 9.
For the adult population over which each product in the array is intended to fit, the cumulative frequency of navel to vulva can also be determined. This is shown in FIG. 10.

For adult females, the vaginal opening can range from between 5 cm to 10 cm from the leading edge of the vulva. However it may be desirable for the acquisition/distribution system to cover the leading edge of the vulva as fluid can run along the body prior to exiting the body and entering the product. For each package in the array, the most stringent criteria for ensuring that the acquisition/distribution system covers the vulva occurs when the navel to article edge is at its lowest (product is at its lowest relative to the navel), coupled with the navel to vulva distance being it's shortest (urine exit point is closer to the navel). Hence the acquisition/distribution system placement from front edge for each product in the array should be designed with this most stringent criteria as the basis. For the S/M package in the above product array example, the shortest navel to vulva distance is approximately 16 cm, and the lowest navel to article edge is approximately −3 cm. Hence the acquisition/distribution system placement from front edge for package S/M embodiment may be set at approximately 13 cm (navel to vulva distance+navel to article edge). This placement will help to ensure acquisition/distribution system coverage over the vulva for the vast majority of adult female consumers within S/M range. The same logic can be applied to determine the placement of the acquisition/distribution system placement for array packages L and XL. This inventive array is shown below in table 2.

TABLE 2
Inventive Array and Examples of Existing Product Arrays

| | Product Array | | | | | |
|---|---|---|---|---|---|---|
| | Assurance Underwear | Depend for Women | Depend Silhouette | Depend Real Fit | | |
| | for Women | Underwear | for Women Briefs | for Men Briefs | Inventive | Inventive |
| | (Maximum | (Maximum | (Maximum | (Maximum | Array | Array |
| | Absorbency) | Absorbency) | Absorbency) | Absorbency) | # 1 | # 2 |
| Number of Packages in Array | 3 | 3 | 2 | 2 | 3 | 2 |
| First Package in Array | S/M | S/M | S/M | S/M | S/M | S/M |
| Product Pitch (cm) | 70 | 72 | 67 | 67 | 72 | 67 |
| AA-FF | | | | | | |
| Absorbent Core Placement (cm) | 13 | 12 | 15 | 8 | 8 | 15 |
| AA-BB | | | | | | |
| Acquisition/Distribution System | 21 | 19 | 20 | 10 | 13 | 13 |
| Placement from Front Edge (cm) | | | | | | |
| AA-CC | | | | | | |
| Absorbent Core Length (cm) | 43 | 41 | 41 | 41 | 49 | 41 |
| BB-EE | | | | | | |
| Acquisition/Distribution System | 21 | 25 | 25 | 25 | 26 | 25 |
| Length (cm) | | | | | | |
| CC-DD | | | | | | |
| Targeted Waist Range (cm) | 71-102 | 71-102 | 71-102 | 71-102 | 71-102 | 71-102 |
| Predicted Urine Exit Point from | 12-30 | 12-30 | 12-30 | 5-23 | 12-30 | 12-30 |
| Navel (cm) | | | | | | |
| Minimum Urine Exit Point to | −9 | −7 | −8 | −5 | −1 | −1 |
| Acquisition/Distribution System | | | | | | |
| Front Edge (AA-CC) (cm) | | | | | | |
| Acquisition/Distribution System to | 30.0% | 26.4% | 29.9% | 14.9% | 18.1% | 19.4% |
| Product Pitch Ratio | | | | | | |
| Second Package in Array | L | L | L/XL | L/XL | L | L/XL |
| Product Pitch (cm) | 75 | 79 | 79 | 79 | 79 | 79 |
| AA-FF | | | | | | |
| Absorbent Core Placement (cm) | 17 | 17 | 19 | 12 | 16 | 19 |
| AA-BB | | | | | | |
| Acquisition/Distribution System | 24 | 24 | 23 | 15 | 16 | 16 |
| Placement from Front Edge (cm) | | | | | | |
| AA-CC | | | | | | |
| Absorbent Core Length (cm) | 43 | 41 | 41 | 41 | 49 | 41 |
| BB-EE | | | | | | |
| Acquisition/Distribution System | 21 | 25 | 25 | 25 | 26 | 25 |
| Length (cm) | | | | | | |
| CC-DD | | | | | | |
| Targeted Waist Range (cm) | 97-127 | 97-127 | 97-127 | 97-127 | 97-127 | 97-127 |
| Predicted Urine Exit Point from | 15-35 | 15-35 | 15-35 | 10-28 | 15-35 | 15-35 |
| Navel (cm) | | | | | | |
| Minimum Urine Exit Point to | −9 | −9 | −8 | −5 | −1 | −1 |
| Acquisition/Distribution System | | | | | | |
| Front Edge (cm) | | | | | | |
| Acquisition/Distribution System to | 32.0% | 30.4% | 29.1% | 19.0% | 20.3% | 20.3% |
| Product Pitch Ratio | | | | | | |
| Third Package in Array | XL | XL | na | na | XL | na |
| Product Pitch (cm) | 80 | 82 | na | na | 82 | na |
| AA-FF | | | | | | |
| Absorbent Core Placement (cm) | 14 | 18 | na | na | 20 | na |
| AA-BB | | | | | | |
| Acquisition/Distribution System | 20 | 24 | na | na | 20 | na |
| Placement from Front Edge (cm) | | | | | | |
| AA-CC | | | | | | |
| Absorbent Core Length (cm) | 52 | 41 | na | na | 49 | na |
| BB-EE | | | | | | |
| Acquisition/Distribution System | 25 | 25 | na | na | 26 | na |
| Length (cm) | | | | | | |
| CC-DD | | | | | | |
| Targeted Waist Range (cm) | 122-163 | 122-163 | na | na | 122-163 | na |
| Predicted Urine Exit Point from | 19-37 | 19-37 | na | na | 19-37 | na |
| Navel (cm) | | | | | | |
| Minimum Urine Exit Point to | −1 | −5 | na | na | −1 | na |
| Acquisition/Distribution System | | | | | | |
| Front Edge (cm) | | | | | | |
| Acquisition/Distribution System to | 25.0% | 29.3% | na | na | 24.4% | na |
| Product Pitch Ratio | | | | | | |
| Array Acquisition/Distribution | 29.0% | 28.7% | 29.5% | 17.0% | 20.9% | 19.8% |
| System to Product Pitch Ratio | | | | | | |

For each of the existing product arrays, it can be seen that the acquisition/distribution system placement from front edge is not adequate to cover all of the intended population.

Identical or Substantially Identical Chassis

As disclosed in U.S. Pub. No. 2013-0211355, it may be desirable to offer an array of packages for fitting different sized wearers, but comprising identical or substantially identical chassis. For instance, an array may comprise a first package comprising a first size of absorbent articles and a second package may comprise a second size of absorbent articles, where the first and second packages comprise identical or substantially identical chassis as described in U.S. Pub. No. 2013-0211355. More particularly, the first package may comprise a first chassis and the second package may comprise a second chassis, where each of the first and second chassis comprise the same dimensions of one or more of: core width at the lateral centerline, core width at one of the front or rear core end, a distance from a left outer cuff distal edge to a right outer cuff distal edge, a distance from a left inner cuff distal edge to a left outer cuff distal edge, a distance from a left inner cuff proximal edge to a right inner cuff proximal edge, a distance from a left inner cuff proximal edge to a left outer cuff distal edge, a free height of the inner cuff, inner cuff hem fold width, inner cuff elastics length, outer cuff elastics length, core length, and backsheet width.

Further, each of the first and second chassis may comprise identical chemical compositions of one or more of a topsheet, backsheet film, backsheet nonwoven, core super absorbent polymers, core pulp, core nonwoven, core tissue, leg cuff film, leg cuff nonwoven, super absorbent polymer adhesive, core nonwoven adhesive, leg cuff elastic adhesive, and backsheet nonwoven/film adhesive.

And, each of the first and second chassis may comprise the same basis weight of one or more of the topsheet, backsheet film, backsheet nonwoven, core super absorbent polymers, core pulp, leg cuff nonwoven, leg cuff film, super absorbent polymer adhesive, leg cuff adhesive, and backsheet nonwoven/film adhesive.

And, each of the first and second chassis may comprise compositionally identical core super absorbent polymers. The first and second chassis may have identical component cross sectional order and disposition in at least one of the front waist region, back waist region, and crotch region. The inner leg cuffs of the first and second chassis may be composed of the compositionally identical materials.

And, the core adhesives of the first and second chassis may be the same adhesive(s). The first and second chassis may comprise core super absorbent polymers that are in the same chemical class and subclass.

And, each of the first and second chassis may comprise first and second wetness indicators, respectively, and wherein the first and second wetness indicators are compositionally identical.

Further, the inner leg cuffs of the first and second chassis may have identical component cross sectional order and disposition in at least one of the front waist region, back waist region, and crotch region. The distance from the left outer cuff distal edge to a right outer cuff distal edge may the same. The distance from the left inner cuff proximal edge to left outer cuff distal edge may be the same. The distance from the left inner cuff proximal edge to the right inner cuff proximal edge is the same. The lengths of the inner and outer cuffs are the same.

### Test Methods

### Product Pitch

The product pitch for each size in the array is determined by:
The testing is performed under ambient room conditions (temperatures from between 15° C. to 35° C. and relative humidity from between 35% to 75%). Samples are conditioned for at least two hours prior to testing under the same conditions. Linear length measurements for sample preparation are made with a device that is traceable to NIST or other standards organization.
Product pitch is defined by the rectangular region defined longitudinally by the first 134 and second 138 waist edges of the product, and laterally by a 10 cm±0.1 cm. The sample cut using sharp scissors and is centered in the product, bisected by L1.
The sample is hung vertically with the product pitch being defined by the distance between the first 134 and second 138 waist edges.
10 products are tested and the product pitch is the average
   Acquisition/Distribution System Placement from Front Edge
Using the same sample from the Product Pitch Measurement, measure the distance from the first 134 waist edge, to the leading edge of the acquisition/distribution system 208.
10 products are tested and the acquisition/distribution system placement from front edge is the average.

### Acquisition/Distribution System to Product Pitch Ratio

The acquisition/distribution system to product pitch ratio is calculated by dividing the acquisition/distribution system placement from front edge by the product pitch and expressing the result as a percentage.
10 products are tested and the acquisition/distribution system to product pitch ratio is the average

### Array Acquisition/Distribution System to Product Pitch Ratio

Is calculated by the average “acquisition/distribution system to product pitch ratio” from all products in an array.

The dimensions and values disclosed herein are not to be understood as being strictly limited to the exact numeral values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of packages comprising three or more different sizes of absorbent articles, the array comprising:
a first package comprising a first absorbent article having a first size and a first waist region;
a second package comprising a second absorbent article having a second size and a second waist region;
a third package comprising a third absorbent article having a third size and a third waist region;
wherein the Array Acquisition/Distribution System to Product Pitch Ratio is from 10% to 28%; and
wherein the first, second, and third packages comprise the same brand name and sub-brand name.

2. The array of claim 1, wherein the Array Acquisition/Distribution System to Product Pitch Ratio is from 15% to 28%.

3. The array of claim 1, wherein the Array Acquisition/Distribution System to Product Pitch Ratio is from 15% to 25%.

4. The array of claim 1, wherein the Array Acquisition/Distribution System to Product Pitch Ratio is from 18% to 22%.

5. The array of claim 1, wherein the Array Acquisition/Distribution System to Product Pitch Ratio is from 19% to 21%.

6. The array of claim 1, wherein the first absorbent article comprises strands in the first waist region and the second absorbent article comprises an elastomeric film in the second waist region.

7. The array claim 1, wherein the first and second absorbent articles comprise compositionally identical core super absorbent polymers.

8. The array claim 1, wherein the first and second absorbent articles of the first and second packages comprise chassis having identical cross-sectional dispositions in at least one of a front, back, or crotch region.

9. The array of claim 1, wherein the array of packages is an on-line array.

10. The array of claim 1, wherein the at least two packages comprise adult incontinence disposable absorbent articles in closed form.

11. An array of packages comprising three or more different sizes of absorbent articles, the array comprising:
a first package comprising a first absorbent article having a first size;
a second package comprising a second absorbent article having a second size;
a third package comprising a third absorbent article having a third size;
wherein the Acquisition/Distribution System Placement from Front Edge is from about 10 cm to about 18 cm for at least one article in the array
wherein the first, second, and third packages comprise the same brand name and sub-brand name.

12. The array of claim 11, wherein the Array Acquisition/Distribution System to Product Pitch Ratio is from 12 cm to 16 cm.

13. The array of claim 11, wherein the Array Acquisition/Distribution System to Product Pitch Ratio is from 13 cm to 15 cm.

14. An array of packages comprising two or more different sizes of absorbent articles, the array comprising:
a first package comprising a first absorbent article having a first size;
a second package comprising a second absorbent article having a second size;
wherein the Acquisition/Distribution System Placement from Front Edge is from about −4 cm to about 2 cm from the predicted urine exit point for the first package in the array; and
wherein the Acquisition/Distribution System Placement from Front Edge is from about −4 cm to about 2 cm from the predicted urine exit point for the second package in the array.

15. The array of claim 14, wherein the first and second absorbent articles comprise compositionally identical core super absorbent polymers and compositionally identical core adhesives.

16. The array of claim 15, wherein the first absorbent article comprises a first chassis and the second absorbent article comprises a second chassis, wherein inner leg cuffs of the first and second chassis may have identical component cross sectional order and disposition in at least one of a front waist region, back waist region, and crotch region.

17. The array of claim 16, wherein the Array Acquisition/Distribution System to Product Pitch Ratio is from 15% to 28%.

18. The array of claim 16, wherein the Array Acquisition/Distribution System to Product Pitch Ratio is from 15% to 25%.

19. The array of claim 16, wherein the Array Acquisition/Distribution System to Product Pitch Ratio is from 18% to 22%.

20. The array of claim 16, wherein the Array Acquisition/Distribution System to Product Pitch Ratio is from 19% to 21%.
